(19) United States
(12) Patent Application Publication
(10) Pub. No.: US 2021/0290131 A1
(43) Pub. Date: Sep. 23, 2021

(54) WEARABLE REPETITIVE BEHAVIOR AWARENESS DEVICE AND METHOD

(71) Applicant: HabitAware, Saint Louis Park, MN (US)

(72) Inventors: Sameer Kumar, Saint Louis Park, MN (US); Aneela Idnani, Saint Louis Park, MN (US); John Pritchard, Saint Louis Park, MN (US); Kirk Klobe, Saint Louis Park, MN (US)

(21) Appl. No.: 17/249,963

(22) Filed: Mar. 19, 2021

Related U.S. Application Data
(60) Provisional application No. 62/992,347, filed on Mar. 20, 2020.

(51) Int. Cl. A61B 5/16; A61B 5/00; A61B 5/11; A61B 5/024; A61B 5/053; G16H 20/60; G16H 20/70; G16H 40/67

(52) U.S. Cl. CPC A61B 5/165; A61B 2562/0219; A61B 5/0075; A61B 5/1118; A61B 5/024; A61B 5/053; A61B 5/0004; A61B 5/746; A61B 5/742; A61B 5/0022; A61B 5/7405; G16H 20/60; G16H 20/70; G16H 40/67; A61B 5/681

(57) ABSTRACT

The present disclosure relates generally to an awareness enhancement apparatus and method for undesirable repeated behaviors, including but not limited to obsessive compulsive and related disorders, and most relevant to trichotillomania (hair pulling), onychophagia (nail biting), dermatillomania (skin picking), and thumb sucking, among others. More particularly, the disclosure relates to a sensing and feedback device and associated methods of use which indicate a behavior based on the user's physical gestures and positioning of the hands, these gestures and positions being related to these undesirable behaviors typical of such disorders, and alerting the user so that he or she can reduce the behavior.

### RELATED APPLICATION

The present application claims the benefit of U.S. Provisional Application No. 62/992,347 filed Mar. 20, 2020, which is hereby incorporated herein in its entirety by reference.

### TECHNICAL FIELD

The present disclosure relates generally to an awareness enhancement apparatus and method for undesirable repeated behaviors, including obsessive compulsive and related disorders. More particularly, the disclosure relates to a sensing and feedback device and associated methods of use which indicates a behavior based on the user's physical gestures related to these undesirable behaviors and alerting the user so that he or she can reduce the behavior.

### BACKGROUND

Nervous behaviors such as trichotillomania (hair pulling), onycophagia (nail biting), dermatillomania (skin picking), thumb sucking and others might be labeled dismissively as “bad habits” and are often harmless for the majority of the affected population. There is, however, increasing focus in the medical community on the group of people for whom these behaviors have significant negative psychological or physical consequences. These specific problematic subtype of behaviors are called body focused repetitive behaviors (BFRBs), which is an umbrella term used to describe certain obsessive compulsive and related behaviors that cause damage to one's body or physical appearance. The prevalence rate of BFRBs has been difficult to determine due to being a poorly understood condition from a scientific perspective and often involving individuals who are attempting to hide their condition(s) or who are not consciously aware of when they are engaging in such behavior. Nevertheless, one study in 2002 of 454 university students reported prevalence rate of BFRBs at 13.7% of the population (Teng, Woods, et al.).

Trichotillomania is one type of BFRB and is characterized by recurrent pulling of one's hair, resulting in hair loss. Reliable trichotillomania prevalence estimates suffer from the two problems of many BFRBs: the individuals that have it may attempt to hide the condition, and there have not been a wealth of academic studies. Nevertheless, the range of reported prevalence is between 0.6-4% (Huynh, Gavino) of the population. In individuals with trichotillomania, hair is most commonly pulled from the scalp, eyebrows and eyelashes but can be pulled from anywhere on the body. The patient may pull hair while being conscious of the action (focused pulling) or the action may be a subconscious behavior (unfocused pulling). When the person engages in focused pulling, he or she may feel an urge to pull from a particular area and feels relief once the hair is pulled. In unfocused pulling, the person may be unaware while he or she is pulling hair, and only become aware once he or she sees the pulled hairs or resulting bald spot. Persons with trichotillomania may suffer from distress due to negative social interactions including bullying and harassment from having thinning or baldness on the scalp, eyebrows, eyelashes or other areas. In spite of the distress caused by this condition, the urge to pull, whether focused or unfocused, can be difficult to overcome. Additionally, patients suffering from trichotillomania, in particular, but also other BFRBs often feel a sense of shame, embarrassment, anger or guilt stemming from their condition.

Individuals with BFRBs generally find methods of hiding their condition, and some may seek treatment. Common methods of hiding trichotillomania may include wigs, hats, eyebrow pencils, false eyelashes, or similar cosmetic approaches. The primary methods of treatment of BFRBs are Cognitive Behavior Therapy (CBT), supportive counseling, support groups, hypnosis, medications and combined approaches (Franklin, Zagrabbe). However, the scientific literature supporting the efficacy of these approaches is not well developed, with fewer than 20 randomized controlled trials available to guide treatment choice and implementation (Franklin, Zagrabbe). The current leading method for addressing BFRBs is Cognitive Behavioral Therapy (CBT), whereby individuals learn how to change their thoughts, feelings, and behaviors by working alongside a therapist or professionally trained psychologist. Studies have shown that, when followed through, CBT can be useful in managing and preventing a wide variety of mental disorders (Trich.org). However, relapse rates can be high once the patient stops CBT. Additionally, CBT is not available to everyone as not all psychologists have been trained in treating BFRBs, not all psychologists practice CBT, and this form of therapy can be prohibitively expensive for many individuals.

Other methods of preventing BFRBs and similar conditions have been presented using some form of physical restraints. U.S. Pat. No. 6,093,158, for example, is directed to a system for monitoring an undesirable behavior from the set of bruxism, jaw clenching, or snoring. A variety of sensors can be used, including those to monitor sound from the undesirable behaviors, signals from muscles in and around the mouth, or force on the teeth. The system described involves wearing an apparatus on the head to monitor the conditions, which is undesirable from a user's perspective due to the common desire to hide the condition via the use of discreet wearable apparatuses.

Another patent, U.S. Pat. No. 4,965,553, discusses a device to alert the user when the hand is near the mouth in order to aid in calorie counting. While it may be effective in reminding the user when that person is eating, eating is an action that is necessary for survival and therefore not always undesirable. Creating a negative feedback signal for an undesirable action can be a more effective system.

Finally, in U.S. Pat. No. 6,762,687, a system of alerting the user when he or she is performing certain obsessive-compulsive spectrum disorders is described. The specific embodiments of the system are comprised of two pieces, a sensor worn on the head, neck or chest, as well as an element associated with the arm, hand, or finger. Such a system is overly cumbersome for the application of preventing a user from a behavior, and a system eliminating one of these pieces could be preferable to users seeking to keep the purpose of the apparatus discreet.

### SUMMARY

Thus, a need exists for a method and apparatus that can monitor, provide feedback about, and ultimately assist in controlling BFRBs that substantially eliminates the problems associated with conventional approaches.

In accordance with the present disclosure, the problem of having a discreet device that alerts the user when performing undesirable behaviors is solved by incorporating orientation and/or gesture recognition into a single device worn on the arm, wrist or hand.

Various objects, features, aspects, and advantages of the present disclosure will become more apparent from the following detailed description of embodiments of the disclosure, along with the accompanying drawings in which like numerals represent like components.

### BRIEF DESCRIPTION OF THE DRAWINGS

FIG. 1 is a drawing of an individual equipped with apparatus according to an embodiment.

FIG. 2 is a simplified block diagram of the electronic circuitry applicable to the embodiments disclosed herein.

FIG. 3 is a flowchart showing action of an embodiment of a system when in use.

### DETAILED DESCRIPTION

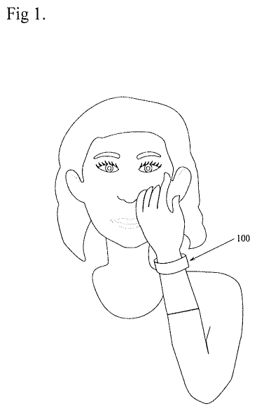

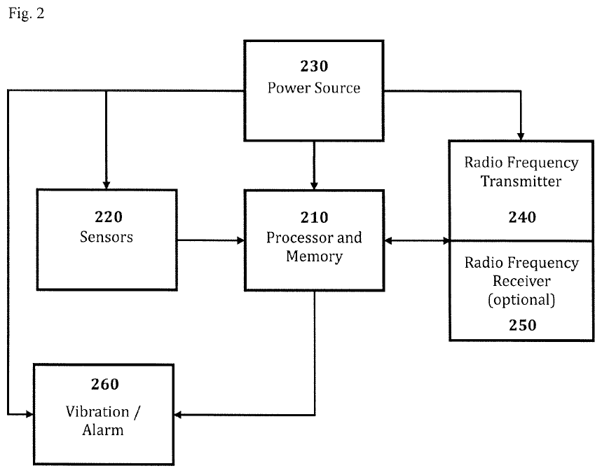

Referring to FIG. 1, a wearable repetitive behavior awareness device 100 is shown in the form of a wrist-band, which includes the components mentioned in FIG. 2. The wearable repetitive behavior awareness device 100 includes a processor and memory 210, sensors 220 (including an inertial measurement unit (IMU) comprised of an accelerometer, gyroscope, and optionally a magnetometer, and may include biofeedback sensors measuring heart rate, skin electrical activity, or other physiological activity), a power source 230, a radio frequency transmitter 240, a radio frequency receiver 250, and a vibration motor or some other real-time tactile, auditory or visual signal 260 to indicate that the bad habit or undesirable behavior has been detected and is occurring.

FIG. 1 shows a user wearing the repetitive behavior awareness device 100 that alerts the user when he or she is performing the undesirable behavior by a tactile, auditory or visual signal. In the preferred embodiment, the alarm is a tactile sensation, such as a vibration, which will allow the device to remain discreet. The device can be trained to actuate the tactile sensation when the user performs a custom gesture or hand orientation associated with a BFRB, and can also come pre-programmed for specific pre-defined common physical gestures and orientations, such as raising the hand to the face and keeping it there.

In one embodiment, the wearable repetitive behavior device is a discreet wrist-worn or hand-worn band, which may have the appearance of a common fitness band or piece of jewelry such as a bracelet or ring. The device sensor unit is an inertial measurement unit accelerometer, gyroscope, and a magnetometer, for optimal hand orientation and gesture recognition. Use of specific biofeedback sensors such as heart rate monitoring and/or skin electrical activity could further augment the accuracy of the device by corroborating biofeedback signals with the inertial measurement unit's readings of orientation and gesture.

In embodiments, a complementary second device may be incorporated according to a user's particular needs. For example, a user using the wearable repetitive behavior device to address an obsessive-compulsive behavior such as repeatedly checking a particular light switch may have the undesired repetitive behavior associated with a particular location. In this example, an RFID or Bluetooth beacon could be used to indicate to the wearable device that a particular pattern of behavior should trigger an alert at the beacon-identified location, but the same pattern of behavior may be inert or even benign if it occurs at a different location. Similarly, a user with disordered eating may have a particular location where their disordered eating primarily occurs, e.g, binge eating may primarily occur adjacent to the user's refrigerator. Such a user may use a beacon to identify the refrigerator to the repetitive behavior awareness device to assist the device in identifying when a binge eating episode is occurring or might soon occur.

In another example, a user's BFRBs may be associated with a heightened emotional state, which may be identified using heart rate variability (HRV). Though HRV can be detected by a hand worn device, such a user may prefer a more sensitive ear clip or patch. An embodiment incorporating such a complementary, alternatively wearable, device also introduces alternative notification options. For instance, an ear clip heart rate monitor (HRM) allows for in-ear notifications. A user may opt for the in-ear complementary device (e.g., earring, headphone(s), ear clip, etc.) for notification purposes only, and without the HRM function. In embodiments, such alternatively wearable devices, rather than a wrist worn device, may be the primary or only device used by a user.

A primary device may be worn on one arm, wrist, or hand, and a secondary device with similar or complementary functionality (but potentially a different form factor, e.g., a ring, earring, necklace, fob, bracelet, pin, clip, band, patch) could be worn on the opposing arm, wrist or hand. This way the user could monitor undesirable behaviors that occur with both hands, as most people with BFRBs and similar conditions use both hands to perform the behavior. The devices are both connected to a single mobile device (e.g. smartphone) via the radio frequency transmitter. For example, a user using the wearable repetitive behavior device to address a nail biting habit may use a wearable device on each hand, so as to receive a prompt notification regardless of which hand is raised to the user's mouth.

### Example Use Case

In the case of an individual with trichotillomania who uses either hand to pull from the eyebrows and eyelashes, a device worn on both wrists would help him or her become more aware of the act of pulling, whenever the algorithm on the device detects the undesired behavior. The user has the option of using the algorithms already programmed on the device (e.g., for common undesirable movements), or can train the algorithm to detect a custom behavior. If the user chooses to train the algorithm, he or she would do so by performing the behavior and giving feedback (details below) so as to minimize the occurrence of false positives (instances when the alarm is actuated but the behavior performed is benign) as well as false negatives (instances when the alarm is not actuated in spite of the undesirable behavior having been performed). Once the algorithm has been trained, the user could wear the device to alert him or her when the hands have moved to the face and are near the eyebrows/eyelashes.

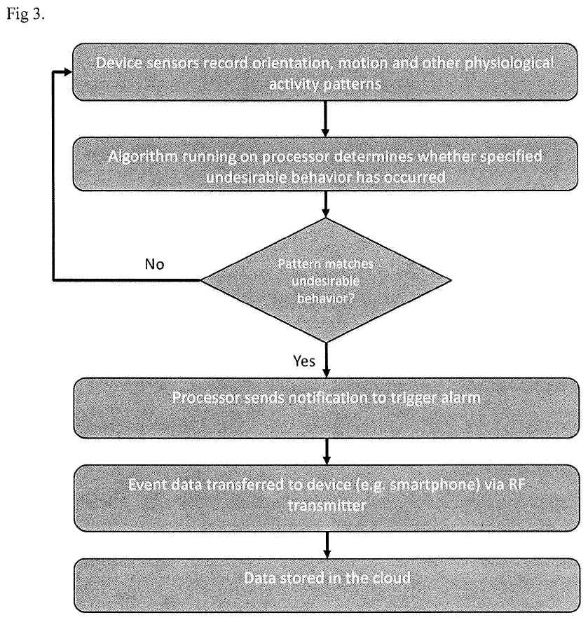

Referring now to FIG. 3, the device would then work as described in the system flowchart 300. The device sensors would record the motion and orientation, at operation 302, and intermittently check if the motion pattern or orientation reading matches that of the trained algorithm, at operation 304. If the processor determines that there is not a match at decision block 306, the device would resume recording at operation 302. If the processor determines there is a match, at decision block 306, then the processor would trigger the alarm, at operation 308, which would preferably be a discreet tactile vibration. The device would record the time that the behavior had occurred and store it in the memory, and transmit the data when connected to the smartphone via the RF transmitter, at operation 310. Finally, the data would be stored in the cloud remotely from the phone for analysis and retrieval in the future, at operation 312.

A benefit of such a device is the real-time feedback via the alarm of the undesirable behavior occurring so that the user can stop him or herself prior to pulling the hair. Additionally, the device is unobtrusive and does not interfere with the user's appearance or normal movements, which would avoid calling attention to the user and the condition so as to increase compliance. Though it may help most during cases of unfocused (subconscious) pulling, the user may derive benefit in cases of focused (conscious) pulling as well because the alert prompts the user to reexamine his or her choice. Over time, and perhaps in conjunction with existing treatments including Cognitive Behavioral Therapy, the alerts from the device could help drive awareness of the behavior, identify the situations that trigger the behavior, and help the user develop strategies for reducing the behavior.

In embodiments, the device may be programmed to deliver predictive alerts, for example by building a machine learning prediction model allowing the device to identify an incipient episode of the BFRB through converging factors such as time, location, activity, mood indicators, etc. The device may be configured to aid the user in averting such an episode, such as through the use of messaging, e.g., “You have a high probability of pulling in the next 30 minutes base on previous data. Do a deep breathing exercise to calm your body and mind.”

Sensor Functionality

The device sensor contains an inertial measurement unit (IMU), including an accelerometer and a gyroscope, and optionally a magnetometer, in some embodiments. The IMU can record specific force, angular rate, and optionally magnetic field data, which can be processed to determine whether a specific motion (i.e. gesture) or hand position (i.e. orientation) is occurring. This information can help the user because in order to perform a BFRB or related behavior, a hand reaches toward another body part such as the head or face. At the end state of this motion, such as in FIG. 1, the orientation of the arm changes and the force of gravity acts on the sensors in a specific and repeatable pattern that can be identified to trigger an alert. Finally, the addition of biofeedback sensors such as heart rate monitors and skin electrical activity sensors are useful to inform when a user is suffering from acute anxiety or stress, which can be correlated to either in progress or imminent BFRB activity. The additional biofeedback sensors improve the accuracy of the device, but are not necessary for the device to perform its basic function of gesture and orientation pattern recognition.

Embodiments may integrate a variety of sensors, and different combinations of sensors may be used depending on a particular user's particular needs and device expectations.

Thermal sensors may use body heat to detect distance away from the body, duration of maintaining a pose (e.g., a user's hand may cool when it has been raised for several minutes), or temperature changes (warmer or cooler) associated with an anxious or fearful state that may be a precursor or a trigger for a BFRB occurrence. In embodiments, a thermal sensor may be configured to detect a user's body temperature, which may in turn be correlated to the user's stress level and stress response. For example, a thermal sensor mounted inside of the wriststrap of a wristworn device would be able to take continuous temperature values of a user's body temperature. This would allow the device to determine correlations between changes in the user's body temperature and occurrence of a BFRB event or other stress response.

In embodiments, a thermal sensor may also or instead be configured to detect the temperature of a surrounding object and evaluate a distance from the user's body accordingly. For example, a sensor positioned on the outside of the strap, on the underside of the wrist, would detect body temperature (e.g., 98° F.) during the most common types of hair-picking BFRB events (e.g., picking of hair from the face or scalp), but would detect ambient temperature (e.g., 70° F.) if a user were to raise their hand, as if in class for instance. The IMU alone might have a difficult time distinguishing between these two behaviors, but the addition of a temperature sensor would improve the device's ability to distinguish between different occurrences of hand raising.

One potential problem with a thermal sensor on a wrist-worn device being used to detect if the hand is touching the body is if a sleeve is being worn over the device, then the thermal sensor will identify the temperature of the sleeve. One way to circumvent this is by using a standard infrared sensor measuring distance to nearest object to identify if the thermal sensor is being covered. This can then aid in only using the thermal sensor when it is not being covered.

A piezo-electric sensor integrated, for example, into a wrist strap (e.g, as a thin film) or ring may identify movement of tendons in the wrist or fingers and thus detect characteristic motions of the fingers, such as picking or pulling. One or more piezoelectric sensor elements can be used in combination with the IMU to improve the accuracy of the finger movement identification. For example, motion signals correlated with the piezoelectric signal can be used to transform the piezoelectric signal artifacts generated when rotating the wrist. Similarly, such a sensor can be combined with near-infrared spectroscopy elements located on the underside of the wrist (integrated into the strap) to enhance finger detection accuracy. For example, an infrared signal can penetrate the skin under the wrist and its reflection can provide information on the wrist tendon location, aiding in the finger movement identification. In the case that the primary device is a patch, the piezoelectric sensor element(s) may also sense vibrations created by speech, heartbeat, or respiration that may indicate when one is performing an unwanted behavior. For example, increase in respiration, heart rate, or heart rate variability may indicate an increase in stress, which is often a precursor to the unwanted behavior. Additionally, unique speech characteristics may also be correlated with the behavior itself or correlated with precursor activities. In the case that the primary device is a patch or in-ear device, the piezo element may sense vibrations due to grinding teeth, helping those who suffer from bruxism. The piezo element may also take advantage of the converse piezo-electric effect, serving as a vibration element (akin to a motor or speaker). For example, if the element is embedded in the strap of a bracelet and a user is performing an undesirable behavior, a voltage can be applied to the piezo-electric element and a vibration can be felt or sound can be heard.

A companion device (which, for example, could be worn as a patch, earring, accessory, ring, bracelet, or integrated into a piece of garment) can be used to send an electrical signal to be received by the primary device (using the skin, clothing, or air as the transmission and reception medium), in which the amplitude or phase (or combination) of the signal indicates the proximity of the primary device to the companion device. The primary device could alert the user, for example, when the hand wearing the companion device is detected near the face, which may aid in reducing an unwanted behavior, such as skin picking of the face. The companion device is comprised of, at least, a power source, oscillator or crystal oscillator, and at least one conductive or semiconductive element that is capacitively coupled with the human skin or garment.

Electrodermal and heart rate variability sensors may be used to identify stress levels and improve the detection algorithm, since skin conductivity and sweat can be correlated to stress. Detection may analyze sweat under and around the device, use an integrated or remote photoplethysmograph, e.g., a wirelessly connected ring, or an integrated or remote electrocardiogram patch. BFRB, OCD, or other negative behaviors may happen more often when a user is stressed. Adding stress detection could improve the certainty of alerts and permit greater sensitivity in distinguishing between negatively-associated and non-negative events and behaviors. Additionally, the user may desire a reminder specifically for stress, as stress on its own is a useful metric to keep track of, not just as an input to the algorithm.

An integrated or remote microphone may be used as a stress sensor, providing an analysis of pitch, tone, and voice control and aligning changes in the user's voice with identified BFRB occurrences.

Continuous glucose monitoring may be provided by an integrated, e.g., a transdermal detector, or communication with a remote device.

Additional sensors for the detection of smoke or gas may also be integrated. For example, a user who uses the device to address a smoking habit may wish to be alerted when smoke in their vicinity reaches a particular threshold. In another example, a user with a checking behavior associated with oven due to intrusive thoughts about a gas in their home may be better able to combat those thoughts if they have an extra layer of security in the form of a wearable gas detector.

Integration of data from a remote device may use a mesh network, e.g., a hip-worn device to provide a point of reference for the primary wrist-worn device, or any of the other possible remote devices discussed herein. The hip-worn device of the preceding example may be especially informative for identifying the location of the wrist-worn device regardless of the user's body orientation, e.g., laying down or standing up. The mesh network may incorporate temporary devices, for example RFID stickers. A user might use such stickers on a daily or almost-daily basis, such as to serve as the hip-worn device of the previous example, or circumstantially, such as when the user is on vacation and wishes to adjust their monitoring system for the change in their routine, such as a user with a checking behavior associated with the oven who wishes to place a beacon near the oven in a vacation house. A mesh network may include multiple devices, and devices with multiple functions. For example, a remote ring device integrated with the network may provide both alerts as well as hand orientation data relative to a wrist-worn or hip-worn device.

Actuating the Vibration Referring again to FIG. 2, the device sensors 220 are connected to a processor 210 that is operative to generate an output signal in the event that the motion or hand orientation being performed by the user matches a particular pre-defined set of undesirable behaviors, which are determined by either the custom training process or a general set of gestures (e.g., raising the hand to the face). The apparatus further includes a device operative to alert the user in response to the output signal generated by the device sensors 220. The device operative to alert the user in response to the output signal generated by the device sensors produces an audible, visual or tactile vibration sensation 260. The sensor housing itself may produce the alert directly, or circuitry may be provided to produce a wireless signal, e.g., via radio frequency transmitter 240, to a separate unit operative to generate an audible, visual or tactile sensation.

Additionally, further functionality is provided to minimize false alarms including appropriate hand orientation and/or gesture recognition, physiological activity, time spent performing an appropriate gesture, contextual information (e.g., if the user is currently using a mobile device) or other behaviors that do not represent any of the undesirable behaviors. The system is also equipped with a manual user-operable override (see “Feedback Mechanisms” section below) to prevent the alarm from being activated for a predetermined period of time to permit acceptable activities (e.g. in the case of hair pulling, the user may want to override the alarm while he or she is eating, which may have a similar motion and hand orientation to hair pulling).

In embodiments, the device may be preprogrammed with a differentiation profile to distinguish between a BFRB behavior and a more inert behavior with a similar hand motion, such as eating or using a cell phone. The device may further be programmed to identify common repetitive behaviors which a user is unlikely to find problematic, such as typing or use of a computer mouse. The differentiation profile may identify a number of corresponding characteristics to permit the device to classify a particular instance of the trigger motion as a BFRB occurrence or an inert occurrence. For example, the duration of repetition of the motion, the ultimate proximity of the user's hand to their face (detected, e.g., by a thermal sensor), the user's associated stress level (detected, e.g., using electrodermal or heart rate measurements), the user's location (detected, e.g., using an RFID device or GPS), etc.

Training Algorithms

There are a number of different gestures associated with one or more undesirable behaviors that users may want to eliminate. For example, in the case of a user who has trichotillomania, the user can pull from the eyebrows and eyelashes or different areas of the scalp, which may likely have different motion patterns and positions of the hands associated with them. To achieve these goals, the user initially calibrates the device with his or her undesirable motions. The wearable repetitive behavior awareness device will record the data associated with the motions from the device sensors 220 in the memory 210 and use that set of data so that the alarm 260 (e.g. vibration motor) will be actuated whenever the user performs the custom motion. The device “gesture training” will impart the advantage of personal customization to detect the undesirable repetitive behaviors.

In embodiments, the user may additionally opt to train the device to differentiate between the negative BFRB behaviors and other, positive or inert, behaviors that may trigger the alarm. For example, a user who uses the device to monitor nail-biting behavior may wish to train the device to differentiate between nail-biting and eating, as both actions may trigger the alarm by using a common hand-to-mouth movement. By indicating to the device when an alarm has been triggered by a non-BFRB behavior, e.g., by silencing or dismissing the alarm, the device may accumulate sufficient data to develop a differentiation profile between the BFRB and the inert behavior. The differentiation profile may include, by example, duration of the hand motion, indicators of increased stress occurring in association with the motion, different characteristic associated motions (such as via a piezo-electric sensor), increased glucose or other biosignature change, etc. In embodiments, the device may prompt the user to either acknowledge the BFRB behavior has occurred or indicate an occurrence of a false alarm.

Embodiments of this disclosure may be configured to provide predictive alerts according to detected time, location, activity, and other factors associated with a user's typical BFRB occurrences. These inputs may be used to create a machine learning prediction model, by the device itself or in communication with a remote application, such as a mobile phone app, to permit the device to determine with a BFRB occurrence may be imminent and alert the user and assist the user in averting the BFRB occurrence.

Mobile Interface—Mobile Phone App, Snooze

In one embodiment, the wearable repetitive behavior awareness device pairs with a mobile device, such as a smartphone or smartwatch to provide the user with additional features and functionality. The features provided with the mobile application include data logging and tracking, amongst others. The user would be able to see data pertaining to their undesirable behavior(s) including when and how often they have performed the behaviors.

In embodiments, the device or the application may be configured to provide notifications to a caregiver or stakeholder. A user may opt to share their progress with their doctor, friends, or family, and thus receive timely support messages from the important people in their lives. The application may be integrated or otherwise connected with a telemedicine platform, creating connections between patients with BFRBs and their doctors, and allowing patient to port relevant data directly to their healthcare professionals. The application may be further integrated or connected with other healthcare or otherwise relevant applications. For example, the application may include sleep tracking features, or receive sleep tracking data from another source, and use sleep data to further develop a predictive profile for BFRB behaviors or other negative behaviors. The application may have similar functionality or connection for tracking of blood sugar levels and determining whether any predictive relationship exists between sugar levels and negative behaviors.

The application may include a variety of features, including but not limited to:
Login incorporating social media and permitting use of a nickname for anonymity.
Tracking, including, for example, the ability to track streaks or records, the ability to set a goal of number of minutes, hours, or days a user wants to try to go behavior free, the ability to add if there was a behavior (“I did”), near behavior (“I wanted” or “I felt the urge”), or a false positive or negative recorded, the ability to add a reason to the record associated with an event; a counter; a reset feature; the ability to add pictures or otherwise to visually track progress; the ability to mark moods (happy, sad, anxious, etc.); the ability to include journal notes (“log for your thoughts”); the ability to track triggers associated with occurrences or to otherwise learn from the good days and the bad days; a visual representation of hair growth, e.g., a seed become a flower becomes a tree.
Community features, including, for example, social posting or commenting; finding or matching with other users for one-to-one support; sharing successes within the application network; sharing successes using social media or email.
Map features, such as links to local groups, e.g., a meetup.org group.
Positive encouragement features, including, for example, positive affirmation control, e.g., speak or type an affirmation to stop an alarm; encouraging texts or quotes after failures or successes; the ability to add images to a vision board of what a user wants, e.g., photos of loved one, or something they are saving up for or an event coming up, or pictures of person with hair as a motivation tool.
Soothing mechanisms and replacement behavior features, including, for example, daily mini activities, e.g., games; coached breathing; short physical exercises or relaxation exercises; daily strength quotes; tips or strategies from psychologists, e.g., change you environment; tips or strategies from other users.
Monetary encouragement features, such as a feature following a gym pact model, where a user may earn cash for streak without a BFRB occurrence or pay up when they have a BFRB occurrence.

Other features may include, for example: a pedometer; a notification pushed to other connected devices; an Android version; Featured Users' Stories to celebrate successes and provide encouragement to others or have them share tips; BFRB Psychologists look up by location; a Positive Affirmation Alarm Clock to start the day right and remind user to wear their repetitive behavior awareness device(s); reminders to focus on the positive, or what you want (e.g., growing hair back) not what you do not want (e.g., to stop pulling).

Feedback Mechanisms

The user is able to deliver feedback to the device directly, via either buttons or physical gestures, or indirectly via the mobile application.

In the direct feedback case, for example, after receiving an alert from the device due to an undesirable behavior detection, the user can confirm the correct reading from the device using a button or through the accelerometer by tapping the device in a predefined way (e.g. tapping twice). Alternatively, the user can inform the device that the behavior was benign by a similar mechanism.

In the indirect feedback case, the user confirms or rejects readings from the device via the mobile app. For example, the mobile app logs each instance that it registers the undesirable behavior with a timestamp, and the user may confirm or correct the readings through the mobile app.

In embodiments, the device may be configured to receive feedback in the form a particular preprogrammed or machine-learned gesture. For example, a user may opt to use the device to assist in habit reversal training, which involves both carefully indentifying triggers and emotional responses associated with BFRB occurrences and deliberate counter BFRB behaviors. A common habit reversal gesture for hair-pulling is to draw the hands into fists at the user's sides. Thus, a user who performs such a habit reversal gesture may program their device to interpret the gesture as positive feedback that a BFRB occurred or was imminent.

Customizable Across Conditions

The present disclosure provides a number of examples focused on common BFRB problems like hair-pulling and nail-biting, but many people exhibit other repetitive, problematic behaviors and may benefit from similar passive monitoring and alerts concerning the behavior. Depending on the specifics of the condition, some important distinctions may exist:

Overeating

Detection: Device may be pre-programmed to detect eating behavior from wrist motion and an optional RFID/Bluetooth Beacon to detect proximity to a given location (e.g., the kitchen in general or the refrigerator specifically).

Notification: User may indicate time of day, duration of eating, and locations of eating before being alerted about binge eating episode.

Predictive alerts: User gets alerts in advance of predicted episodes based on user's behavior (e.g., indicators or rising stress levels, multiple trips to the kitchen detected over a short period of time, etc.).

Smoking

Detection: Device may be pre-programmed to detect smoking behavior from wrist motion.

Notification: User may receive notification after X number of cigarettes per day or other specified period of time.

Predictive alerts: User may receive alerts in advance of predicted episodes based on user's behavior patterns (e.g., device detects user repeatedly reaching for their pocket where they carry cigarettes, user frequently smokes at certain times of day, locations, etc.).

Anxiety

Detection: Device may use any of pre-programming, user training, or some combination of the two to detect a panic attack or otherwise anxious state. Device may use electrodermal sensors, heart rate monitoring, motion sensors, etc to determine the user's stress level and response.

Notification: User may be notified during a panic attack and given instructions to assist them in coping (e.g., the device recommends the user find a quiet place to sit, provides a guided deep breathing or counting exercise, etc.)

Predictive alerts: User may receive alerts in advance of predicted episodes based on the user's past behavior.

Attention-Deficit Hyperactivity Disorder (ADHD)

Detection based on motion and physiological parameters: Device captures user's baseline of hand or physical motion, or heart rate, over several weeks, and calculates a score based on aggregate levels of ADHD activity. This feature may be of particular interest for doctors to objectively decide on medication dosing and changes.

Notifications: User may be notified during high levels of ADHD-related behavior.

Autism

Detection: Device may be pre-programmed to detect flapping or other stimming behavior from motion.

Notification: User and caregiver may be notified and given strategies during the episode.

OCD

Detection: Device may be pro-programmed, trained by user, or some combination thereof to detect checking behaviors (e.g. hand washing, oven, light switch, etc.). Use of RFID/Bluetooth beacon may be incorporated for users with behaviors associated with particular locations.

Notification: User may receive notifications and real-time strategies via device or smartphone.

Predictive alerts: User sent predictive alerts with strategies based on user's previous behavior

Intrusive Thoughts (Measuring Body's Response to Thoughts)

Detection: Brain signals may be received, e.g., an electroencephalogram (EEG) headband, and correlated to behavior patterns or thoughts. The EEG headband, or other brain signal detection device, may be a user's primary or secondary device. Behaviors and thoughts may be user-identified, or detected by the device, e.g., by incorporating data from other sensors. Intrusive thoughts may also be correlated to other biofeedback signals based on user identification or preprogrammed settings.

Notification: User may receive notifications and real-time strategies via device or smartphone.

Predictive alerts: User sent predictive alerts with strategies based on user's previous behavior

Tremors/Parkinson's

Detection: Device may detect oscillations of the user's limbs.

Notification: Events may be uploaded to a cloud database or used to notify a doctor or caregiver for increased frequency.

While some embodiments of the disclosure have been described in reference to the drawings, the disclosure is not so limited. For example, the device can be used without an alarm feature. In some applications it may be desirable to simply collect information associated with a behavior to determine if a particular treatment has helped, or if the behavior has worsened or improved over time. Thus, the alarm can be turned on or off as needed to both alert the user, and/or merely allow the device to collect information.

Further, the device can be used as a positive feedback device. For example, in the case of BFRBs the device can detect periods when the behavior is absent and emit an alert (such as a pleasant tone) that may assist the user in understanding when the behavior is not occurring, or as a reward.

Further, the device can be used as a feedback mechanism for any physical bad habit that the user may want to track or reduce, which may or may not be classified as BFRBs. Some examples of such habits could be smoking, overeating, or hair twirling. Still further, the device can be used in connection with behaviors that may be repetitive but not necessarily harmful or undesirable. These could be precursor behaviors associated with the onset of BFRBs. Or, the behaviors monitored could have nothing to do with disorders but instead the device could monitor body position relationships that may be positive or negative to a user in the field of sports, ergonomics, and the like.

Unless otherwise defined, all technical and scientific terms used herein have the same meaning as commonly understood by one of ordinary skill in the art to which this disclosure belongs. Although methods and materials similar to or equivalent to those described herein can be used in the practice or testing of the present disclosure, suitable methods and materials are described below. All publications, patent applications, patents, and other references mentioned herein are incorporated by reference in their entirety to the extent allowed by applicable law and regulations. In case of conflict, the present specification, including definitions, will control.

The present disclosure may be embodied in other specific forms without departing from the spirit or essential attributes thereof, and it is therefore desired that the present embodiment be considered in all respects as illustrative and not restrictive, reference being made to the appended claims rather than to the foregoing description to indicate the scope of the disclosure. Those of ordinary skill in the art that have the disclosure before them will be able to make modifications and variations therein without departing from the scope of the disclosure.

What is claimed is:

1. A wearable repetitive behavior awareness device comprising:
a band;
a power source;
one or more sensors comprising:
an inertial measurement unit (IMU) comprising an accelerometer and gyroscope, and
a piezo-electric force sensor configured to detect digit motion;
a memory comprising instructions for operation of the one or more sensors; and
a processor in communication with the memory and the one or more sensors, the processor configured to execute the instructions stored in the memory to detect a localized repetitive behavior by having a user repeat the behavior while the device is in a training mode in which the device stores an electronic representation of the behavior in the memory, and subsequently monitors the user to determine if the localized repetitive behavior is present.

2. The wearable repetitive behavior awareness device of claim 1, wherein the piezo-electric force sensor is a thin film sensor.

3. The wearable repetitive behavior awareness device of claim 1, wherein the band is a wristband.

4. The wearable repetitive behavior awareness device of claim 3, wherein the one or more sensors further comprise near-infrared spectroscopy elements oriented on an underside of the user's wrist and configured to detect wrist tendon location.

5. The wearable repetitive behavior awareness device of claim 3, wherein the device is a first repetitive behavior awareness device; and
the first repetitive behavior awareness device in communication with a second wearable repetitive behavior awareness device.

6. The wearable repetitive behavior awareness device of claim 1, wherein the band is a ring.

7. The wearable repetitive behavior awareness device of claim 1, wherein the IMU further comprises a magnetometer.

8. The wearable repetitive behavior awareness device of claim 1, wherein the IMU further comprises one or more biofeedback sensors configured to measure one or more of heart rate, skin electrical activity, and other physiological activity.

9. The wearable repetitive behavior awareness device of claim 8, wherein the processor is further configured to distinguish between the localized repetitive behavior and other benign activities.

10. The wearable repetitive behavior awareness device of claim 1, wherein the localized repetitive behavior is associated with when the user is smoking.

11. The wearable repetitive behavior awareness device of claim 1, wherein the localized repetitive behavior is associated with when the user is eating.

12. The wearable repetitive behavior awareness device of claim 1, wherein the localized repetitive behavior is associated with when the user is performing a body focused repetitive behavior.

13. The wearable repetitive behavior awareness device of claim 1, further comprising a radio frequency transmitter and a radio frequency receiver providing communication between the processor and a remote application.

14. The wearable repetitive behavior awareness device of claim 1, further comprising an alert element.

15. The wearable repetitive behavior awareness device of claim 14, wherein the alert element generates one or more of a haptic alert, an auditory alert, or a visible alert.

16. The wearable repetitive behavior awareness device of claim 14, wherein the processor is further configured to override the alert when the processor determines that the user is performing a benign movement that mimics the localized repetitive behavior.

17. A system for providing repetitive behavior awareness, comprising:
a first behavior awareness device comprising:
a processor,
a memory, and
sensors for detecting user movements and comprising:
an inertial measurement unit (IMU) comprising an accelerometer and a gyroscope, and
a piezo-electric force sensor configured to detect digit motion; and
a remote computing application interfaced with the first behavior awareness device and configured for storing information collected by the sensors and performing behavior tracking.

18. The system for providing repetitive behavior awareness of claim 17, further comprising a second behavior awareness device interfaced with the first behavior aware device and the remote computing application.

19. The system for providing repetitive behavior awareness of claim 18, wherein the first behavior awareness device is worn on a first wrist of a user and the second behavior awareness device is worn on a second wrist of the user.

20. A repetitive behavior awareness method, comprising:
providing a behavior awareness device comprising a computer processor, a computer memory, sensors for detecting user movements, wherein the sensors comprise an inertial measurement unit (IMU) comprising an accelerometer and gyroscope, and a piezo-electric force sensor configured to detect digit motion;
programing the device to detect a localized repetitive behavior, the localized repetitive behavior comprising the detected digit motion, by having a user repeat the behavior while the behavior awareness device is in a training mode in which the behavior awareness device stores an electronic representation of the localized repetitive behavior in the memory; and
monitoring a user with the behavior awareness device to determine if the programmed localized repetitive behavior is present by using an algorithm stored in the memory to compare user behavior to the localized repetitive behavior stored in the memory during training.
